(19) United States
(12) Patent Application Publication
(10) Pub. No.: US 2022/0334714 A1
(43) Pub. Date: Oct. 20, 2022

(54) METHOD AND SYSTEM FOR PRESENTING DYNAMICALLY UPDATED VISUAL FEEDBACK AT A MAIN DISPLAY BASED ON TOUCH PANEL CONTROL INTERACTION

(71) Applicant: GE Precision Healthcare LLC, Wauwatosa, WI (US)

(72) Inventors: Carolyn Sweet, Vöcklamarkt (AT); Andreas Haas, Schoerfling am Attersee (AT); Balint Czupi, Seewalchen am Attersee (AT)

(21) Appl. No.: 17/231,241

(22) Filed: Apr. 15, 2021

(51) Int. Cl. G06F 3/0488; G06F 3/0484; G06F 3/041; A61B 8/00

(52) U.S. Cl. CPC G06F 3/04886; G06F 3/04847; G06F 3/041; A61B 8/465; A61B 8/467; G06F 2203/04108; G06F 3/04842; G06F 2203/04803

(57) ABSTRACT

A system and method for detecting user interaction with a touch panel control of an ultrasound system and providing visual feedback at a main display identifying the control corresponding to the user interaction is provided. The method includes receiving a detection signal from a touch panel in response to detection of a user in proximity to the touch panel. The detection signal corresponds with a location on the touch panel associated with a touch panel control. The method includes processing the detection signal to display a mirrored representation of the touch panel control in a dedicated area of the main display of the display system. The method includes receiving an actuation signal from the touch panel in response to a user selection at the touch panel. The method includes processing the actuation signal to adjust a setting value of the ultrasound system associated with the touch panel control.

### FIELD

Certain embodiments relate to medical imaging, and particularly ultrasound imaging. More specifically, certain embodiments relate to a method and system for detecting user interaction with a touch panel control of an ultrasound imaging system and providing visual feedback at a main display identifying the control and associated setting value corresponding to the user interaction with the touch panel control.

### BACKGROUND

Ultrasound imaging is a medical imaging technique for imaging organs and soft tissues in a human body. Ultrasound imaging uses real time, non-invasive high frequency sound waves to produce a series of two-dimensional (2D) and/or three-dimensional (3D) images.

Ultrasound systems typically include an ultrasound scanner, a touch panel, and a main display. An ultrasound operator may manually maneuver the ultrasound scanner on a patient while interacting with the touch panel and viewing the ultrasound image data at the main display during an ultrasound examination. Accordingly, the ultrasound operator may have to repeatedly look away from the main display to locate the appropriate controls presented at the touch panel such that the operator may manipulate or adjust the controls of the ultrasound system during the examination, which may be inefficient. Furthermore, non-image display elements presented at a main display may distract a user trying to review one or more ultrasound images at the main display.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for detecting user interaction with a touch panel control of an ultrasound imaging system and providing visual feedback at a main display identifying the control and associated setting value corresponding to the user interaction with the touch panel control, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable to detect user interaction with a touch panel control and provide visual feedback at a display system identifying the control and associated setting value corresponding to the user interaction with the touch panel control, in accordance with various embodiments.

FIG. 2 is a display of an exemplary main display and touch panel display in a two-dimensional (2D) imaging mode, the main display configured to present a control and associated setting value corresponding to user interaction with the touch panel, in accordance with various embodiments.

FIG. 3 is a display of an exemplary main display and touch panel display in a pulse wave (PW) imaging mode, the main display configured to present a control and associated setting value corresponding to user interaction with the touch panel, in accordance with various embodiments.

FIG. 4 is a display of an exemplary main display and touch panel display in a color flow mapping (CFM) imaging mode, the main display configured to present a control and associated setting value corresponding to user interaction with the touch panel, in accordance with various embodiments.

FIG. 5 is a flow chart illustrating exemplary steps that may be utilized for providing visual feedback at a main display identifying a control and associated setting value corresponding to user interaction with a touch panel control, in accordance with various embodiments.

### DETAILED DESCRIPTION

Certain embodiments may be found in a method and system for detecting user interaction with a touch panel control of an ultrasound system and providing visual feedback at a main display identifying the control and associated setting value corresponding to the user interaction with the touch panel control. Aspects of the present disclosure have the technical effect of providing visual feedback at a dedicated area of a main display mirroring touch panel controls that an operator is interacting with on a touch panel such that the operator does not have to look away from the main display. Certain embodiments have the technical effect of providing visual feedback at a dedicated area on a main display related to a position of buttons or groups of buttons that an operator is interacting with on a touch panel. Various embodiments have the technical effect of providing visual feedback at a dedicated area of a main display of current touch panel control settings values with which an operator is interacting. Aspects of the present disclosure have the technical effect of presenting visual feedback at a dedicated area of a main display only when an operator is interacting with a control on a touch panel such that an operator is not distracted by the non-image display elements when reviewing ultrasound images. Certain embodiments provide the technical effect of providing a dedicated area of a main display that does not include fixed content but rather is dynamically updated in substantially real-time based on interaction with different locations on a touch panel by an operator. Various embodiments provide the technical effect of distinguishing between detected interaction (e.g., hovering over a touch panel control or a light touch of a touch panel control) and actuation (e.g., touch or firm touch of a touch panel control). Aspects of the present disclosure provide the technical effect of mirroring one of a plurality of controls presented at a touch panel at a dedicated area of a main display based on a location of user interaction at the touch panel. Certain embodiments have the technical effect of providing visual feedback at a main display of touch panel control setting value adjustments in response to user interaction at the touch panel.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and proceeded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode such as B-mode (2D mode), M-mode, three-dimensional (3D) mode, CF-mode, CFM-mode, PW Doppler, CW Doppler, MGD, and/or sub-modes of B-mode and/or CF such as Shear Wave Elasticity Imaging (SWEI), TVI, Angio, B-flow, BMI, BMI_Angio, and in some cases also MM, CM, TVD where the “image” and/or “plane” includes a single beam or multiple beams.

Furthermore, the term processor or processing unit, as used herein, refers to any type of processing unit that can carry out the required calculations needed for the various embodiments, such as single or multi-core: CPU, Accelerated Processing Unit (APU), Graphics Board, DSP, FPGA, ASIC or a combination thereof.

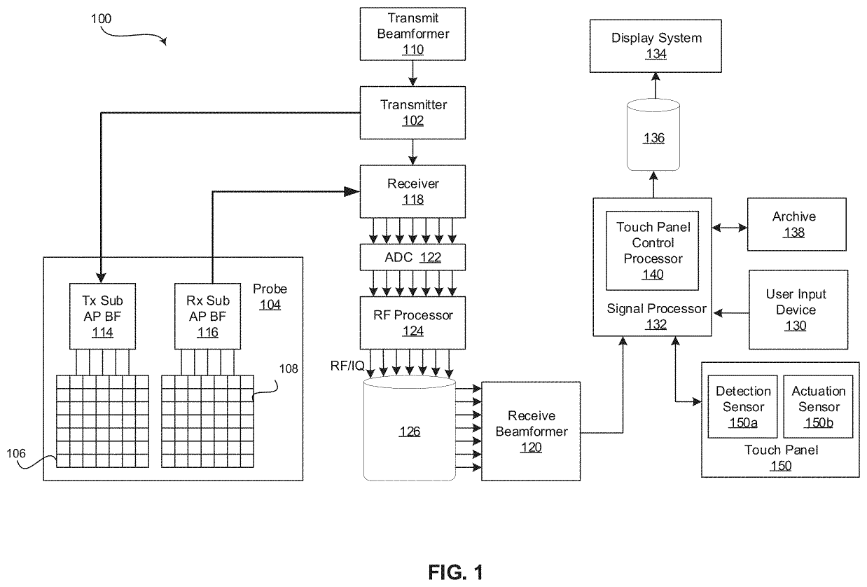

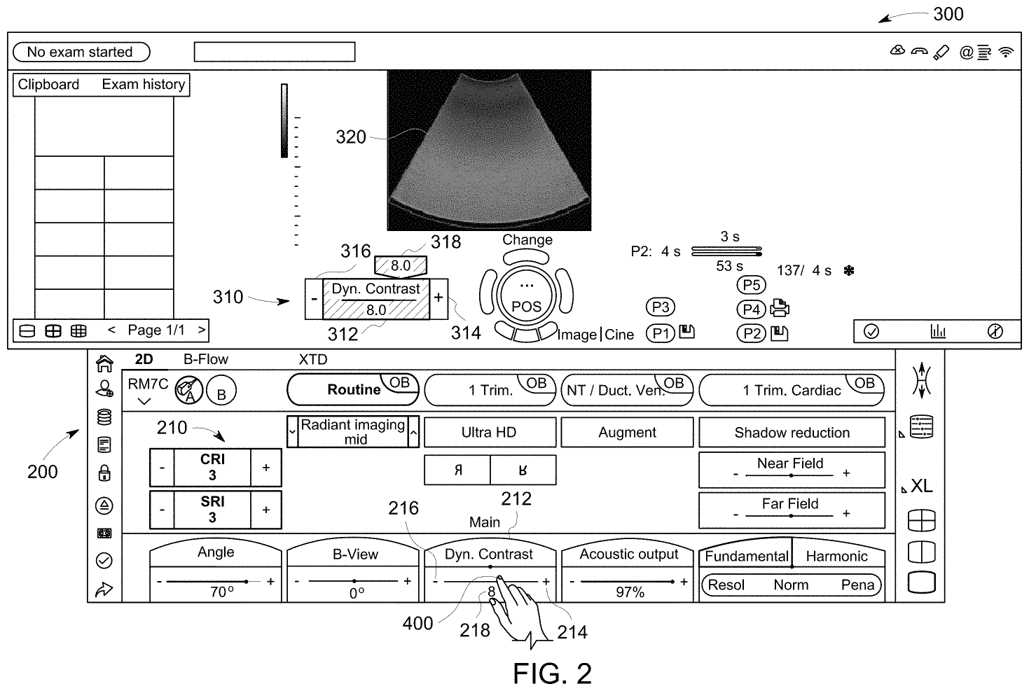

It should be noted that various embodiments are described herein with reference to a touch panel and main display of an ultrasound system. For example, FIG. 1 illustrates an exemplary ultrasound system and FIGS. 2-4 illustrate an exemplary main display and touch panel of an ultrasound system. However, aspects of the present invention are not limited to ultrasound systems. Instead, any medical device having a main display and touch panel is contemplated.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 that is operable to detect user interaction with a touch panel control and provide visual feedback at a display system 134 identifying the control and associated setting value corresponding to the user interaction with the touch panel control, in accordance with various embodiments. Referring to FIG. 1, there is shown an ultrasound system 100. The ultrasound system 100 comprises a transmitter 102, an ultrasound probe 104, a transmit beamformer 110, a receiver 118, a receive beamformer 120, A/D converters 122, a RF processor 124, a RF/IQ buffer 126, a user input device 130, a signal processor 132, an image buffer 136, a display system (also referred to as a main display) 134, an archive 138, and a touch panel 150.

The transmitter 102 may comprise suitable logic, circuitry, interfaces and/or code that may be operable to drive an ultrasound probe 104. The ultrasound probe 104 may comprise a two dimensional (2D) array of piezoelectric elements. The ultrasound probe 104 may comprise a group of transmit transducer elements 106 and a group of receive transducer elements 108, that normally constitute the same elements. In certain embodiment, the ultrasound probe 104 may be operable to acquire ultrasound image data covering at least a substantial portion of an anatomy, such as the heart, a blood vessel, a fetus, or any suitable anatomical structure.

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118. The receiver 118 may comprise suitable logic, circuitry, interfaces and/or code that may be operable to receive the signals from the receive sub-aperture beamformer 116. The analog signals may be communicated to one or more of the plurality of A/D converters 122.

The plurality of A/D converters 122 may comprise suitable logic, circuitry, interfaces and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118.

The RF processor 124 may comprise suitable logic, circuitry, interfaces and/or code that may be operable to demodulate the digital signals output by the plurality of A/D converters 122. In accordance with an embodiment, the RF processor 124 may comprise a complex demodulator (not shown) that is operable to demodulate the digital signals to form I/Q data pairs that are representative of the corresponding echo signals. The RF or I/Q signal data may then be communicated to an RF/IQ buffer 126. The RF/IQ buffer 126 may comprise suitable logic, circuitry, interfaces and/or code that may be operable to provide temporary storage of the RF or I/Q signal data, which is generated by the RF processor 124.

The receive beamformer 120 may comprise suitable logic, circuitry, interfaces and/or code that may be operable to perform digital beamforming processing to, for example, sum the delayed channel signals received from RF processor 124 via the RF/IQ buffer 126 and output a beam summed signal. The resulting processed information may be the beam summed signal that is output from the receive beamformer 120 and communicated to the signal processor 132. In accordance with some embodiments, the receiver 118, the plurality of A/D converters 122, the RF processor 124, and the beamformer 120 may be integrated into a single beamformer, which may be digital. In various embodiments, the ultrasound system 100 comprises a plurality of receive beamformers 120.

The user input device 130 and/or touch panel 150 may be utilized to input patient data, scan parameters, settings, select protocols and/or templates, and the like. In various embodiments, the user input device 130 may be or may include a touch panel 150. In an exemplary embodiment, the user input device 130 and/or touch panel 150 may be operable to configure, manage and/or control operation of one or more components and/or modules in the ultrasound system 100. In this regard, the user input device 130 and/or touch panel 150 may be operable to configure, manage and/or control operation of the transmitter 102, the ultrasound probe 104, the transmit beamformer 110, the receiver 118, the receive beamformer 120, the RF processor 124, the RF/IQ buffer 126, the user input device 130, the signal processor 132, the image buffer 136, the display system 134, and/or the archive 138. The user input device 130 may include a touch panel 150, button(s), rotary encoder(s), motion tracking, voice recognition, a mousing device, keyboard, camera and/or any other device capable of receiving a user directive. In certain embodiments, one or more of the user input devices 130 may be integrated into other components, such as the display system 134, for example. As an example, user input device 130 may include a touch panel 150 or other touchscreen display.

The touch panel 150 may be operable to present selectable controls for controlling operation of the ultrasound system 100. The controls may be selectable and setting values associated with the controls adjustable in response to user touch interaction on a surface of the touch panel 150. The touch panel 150 may comprise a detection sensor 150a and an actuation sensor 150b. In various embodiments, the detection sensor 150a and the actuation sensor 150 may be a same sensor or group of sensors. The actuation sensor 150b may comprise suitable logic, circuitry, interfaces and/or code that may be operable to detect the actuation of the touch panel 150. For example, the actuation sensor 150b may detect the depression of a surface of the touch panel 150. The detection sensor 150a may comprise suitable logic, circuitry, interfaces and/or code that may be operable to detect a touch or close proximity of a user finger to the detection sensor 150a of the touch panel 150. The detection of the user proximity is separate from any subsequent or simultaneous detection of an actuation. The detection of the user proximity and/or actuation may be associated with a location on a surface of the touch panel 150. The locations on the surface of the touch panel 150 may be associated with touch panel controls presented at corresponding positions on the touch panel 150. The detection sensor 150a and/or actuation sensor 150b may be a resistive sensor, capacitive sensor, infrared sensor, or any suitable sensor operable to detect a user touching and/or in close proximity to the sensor. The detection 150a and actuation 150b sensing may be performed by resistive film touch panels, surface capacitive touch panels, projected capacitive touch panels, surface acoustic wave (SAW) touch panels, optical touch panels (e.g., infrared optical imaging touch panels), electromagnetic induction touch panels, or any suitable touch panel. In various embodiments, the touch panel 150 may be configured in a number of ways to distinguish between detection and actuation. For example, detection may correspond with a light touch or hovering over a location of the touch panel 150 and actuation may correspond with a firm touch (e.g., increased pressure or pressure above a threshold) at a location of the touch panel 150. As another example, detection may be associated with a touch input at a location of the touch panel 150 and actuation may be associated with a double touch at a location of the touch panel 150. Another example may include a single finger at a location of the touch panel 150 corresponding with detection and a multi-touch input (e.g., two fingers) may correspond with actuation. In various embodiments, the touch panel 150 may be configurable to define detection sensing functionality and actuation sensing functionality.

The signal processor 132 may comprise suitable logic, circuitry, interfaces and/or code that may be operable to process ultrasound scan data (i.e., summed IQ signal) for generating ultrasound images for presentation on a display system (also referred to as a main display) 134. The signal processor 132 is operable to perform one or more processing operations according to a plurality of selectable ultrasound modalities on the acquired ultrasound scan data. In an exemplary embodiment, the signal processor 132 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation. In various embodiments, the processed image data can be presented at the display system 134 and/or may be stored at the archive 138. The archive 138 may be a local archive, a Picture Archiving and Communication System (PACS), or any suitable device for storing images and related information.

The signal processor 132 may be one or more central processing units, microprocessors, microcontrollers, and/or the like. The signal processor 132 may be an integrated component, or may be distributed across various locations, for example. In an exemplary embodiment, the signal processor 132 may comprise a touch panel control processor 140 and may be capable of receiving input information from user input devices 130 and/or archive 138, generating an output displayable by a display system 134, and manipulating the output in response to input information from a user input device 130 and/or touch panel 150, among other things. The signal processor 132 and touch panel control processor 140 may be capable of executing any of the method(s) and/or set(s) of instructions discussed herein in accordance with the various embodiments, for example.

The ultrasound system 100 may be operable to continuously acquire ultrasound scan data at a frame rate that is suitable for the imaging situation in question. Typical frame rates range from 20-120 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes' worth of frames of ultrasound scan data. The frames of ultrasound scan data are stored in a manner to facilitate retrieval thereof according to its order or time of acquisition. The image buffer 136 may be embodied as any known data storage medium.

The signal processor 132 may include a touch panel control processor 140 that comprises suitable logic, circuitry, interfaces and/or code that may be operable to selectively present, at a dedicated area in a main display of the display system 134, touch panel controls mirrored from the touch panel 150 in response to user interactions with locations on a surface of the touch panel 150. The dedicated area in the main display of the display system 134 may be below an ultrasound image display area or any suitable location on the main display (e.g., on a left side, a right side, or above the ultrasound image display area). In certain embodiments, the dedicated area and the ultrasound image display area are separate and distinct (i.e., non-overlapping) areas of the main display of the display system 134. In various embodiments, a location of the dedicated area on the main display of the display system 134 may be user-configurable. The touch panel control processor 140 may be configured to leave the dedicated area on the main display blank prior to detected user interaction with the touch panel 150 and after a predetermined period of time without user interaction with the touch panel 150 such that an operator is not distracted by the presentation of non-image display elements. For example, prior to user interaction with the touch panel 150, such as when an ultrasound operator is manipulating the ultrasound probe 104 and reviewing the acquired ultrasound images on the display system 134, the dedicated area on the main display of the display system 134 may be left blank. As another example, after an ultrasound operator adjusts setting values via touch panel controls of the touch panel 150 and removes their finger from the touch panel 150, such as to resume manipulation of the ultrasound probe 104 and/or review of the acquired ultrasound images, the touch panel control processor 140 may remove the displayed touch panel control and present nothing in the dedicated area of the main display of the display system 134 after a predetermined period of time (e.g., after 1-5 seconds without user interaction at the touch panel 150).

The touch panel control processor 140 may comprise suitable logic, circuitry, interfaces, and/or code that may be operable to receive a detection signal from a detection sensor 150a of the touch panel 150 and present, in the dedicated area of the main display, the touch panel control corresponding with the location of the detected user interaction on the touch panel 150. For example, the touch panel 150 may present various controls, each having an associated setting value and buttons, sliders, or the like for adjusting the setting value, at various locations on the touch panel 150. The detection sensor 150a may detect a user interaction (e.g., touch input or hovering over a particular control location) and provide a detection signal identifying the location of the detected user interaction to the touch panel control processor 140. The touch panel control processor 140 may be configured to process the detection signal to identify the particular control presented at the location on the touch panel 150 and present a mirrored representation of the particular control in the dedicated area at the main display of the display system 134 such that an ultrasound operator does not have to look away from the main display to visualize the control the user is interacting with at the touch panel 150. For example, the identification of the control, the setting value associated with the control, and the buttons, sliders, or the like for adjusting the setting value of the control may be presented in the dedicated area of the main display in substantially a same manner as presented at the touch panel 150 to provide visual feedback to an ultrasound operator such that the operator is able to interact with the buttons, sliders, and the like to adjust the setting value of the control without looking at the touch panel 150. In an exemplary embodiment, the mirrored representation of the touch panel control includes a positional indicator showing a position of the ultrasound operator (e.g., the ultrasound operator's finger) relative the touch panel control.

The touch panel control processor 140 may comprise suitable logic, circuitry, interfaces, and/or code that may be operable to receive an actuation signal from an actuation sensor 150b of the touch panel 150 and adjust a setting value of the detected control in response to the user actuation of the touch panel 150. For example, an ultrasound operator may actuate a button, slider, or the like on the touch panel 150 based on the visual feedback provided in the dedicated area of the main display of the display system 134. The touch panel control processor 140 receives the actuation signal from the actuation sensor 150b of the touch panel 150 and processes the actuation signal to implement the setting value adjustment. The touch panel control processor 140 dynamically updates the presentation of the setting value at the dedicated area of the main display of the display system 134.

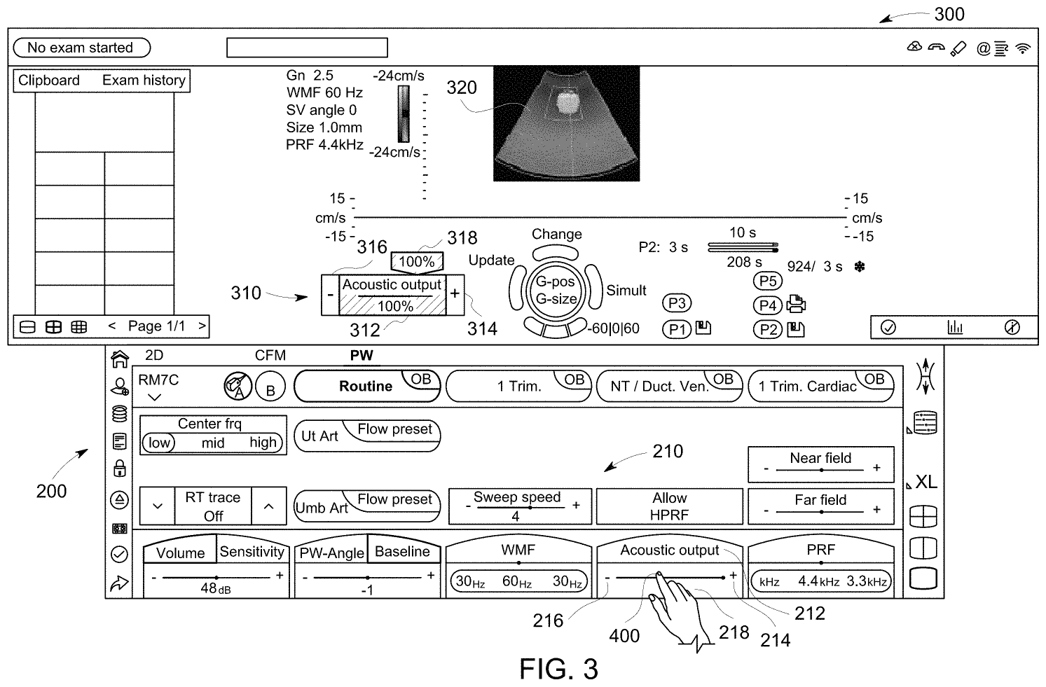

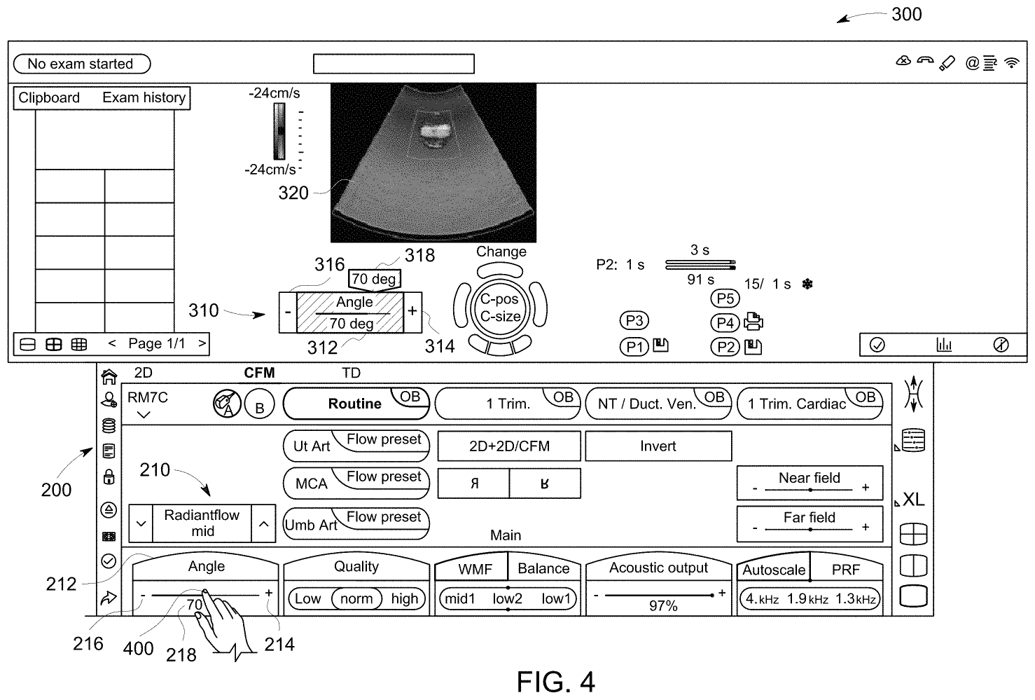

FIG. 2 is a display of an exemplary main display 300 and touch panel display 200 in a two-dimensional (2D) imaging mode, the main display 300 configured to present a control 312 and associated setting value 318 corresponding to user interaction with the touch panel 150, 200, in accordance with various embodiments. FIG. 3 is a display of an exemplary main display 300 and touch panel display 200 in a pulse wave (PW) imaging mode, the main display 300 configured to present a control 312 and associated setting value 318 corresponding to user interaction with the touch panel 150, 200, in accordance with various embodiments. FIG. 4 is a display of an exemplary main display 300 and touch panel display 200 in a color flow mapping (CFM) imaging mode, the main display 300 configured to present a control 312 and associated setting value 318 corresponding to user interaction with the touch panel 150, 200, in accordance with various embodiments.

Referring to FIGS. 2-4, the main display 400 may be the display of the display system 134 of FIG. 1. The touch panel display 200 may be the display of the touch panel 150 of FIG. 1. The touch panel display 200 may comprise controls 210 operable to adjust setting values 218 of an ultrasound examination. For example, in a 2D imaging mode as shown in FIG. 2, the controls 210 may include a crossbeam imaging (CRI) setting value, a speckle reduction imaging (SRI) setting value, an angle setting value, a dynamic contrast 212 setting value 218, an acoustic output setting value, fundamental and harmonic setting values, near field and far field setting values, and the like. As another example, in a PW imaging mode as shown in FIG. 3, the controls 210 may include volume and sensitivity setting values, PW angle and baseline setting values, a wall motion filter (WMF) setting value, a pulse repetition frequency (PRF) setting value, an acoustic output 212 setting value 218, a real time (RT) trace setting value, and the like. As another example, in a color flow mapping (CFM) imaging mode as shown in FIG. 4, the controls 210 may include an angle 212 setting value 218, a quality setting value, wall motion filter and balance setting values, an acoustic output setting value, auto scale and pulse repetition setting values, near field and far field setting values, a radiant flow setting value, and the like. The controls 210 may each include an identifier 212 of the control, a current setting value 218, and buttons 214, 216, sliders, or the like for increasing 214 or decreasing 216 the setting value. The controls 210 may be manipulated by a user finger 400 actuating the buttons 212, 214, sliders, or the like presented at the touch panel display 200 of the touch panel 150.

Still referring to FIGS. 2-4, the main display 300 may include an ultrasound image display area configured to present an ultrasound image 320 and a dedicated area 310 configured to selectively present visual feedback 312-318 related to user interaction and actuation of touch panel controls 210. The visual feedback 312-318 may be presented in the dedicated area 310 of the main display 300 in response to user interaction 400 with a touch panel display 200 and may mirror the touch panel controls 210 presented and interacted 400 with at the touch panel display 200 of the touch panel 150. For example, the visual feedback may include an identifier 312 of the control interacted with at the touch panel 150, a current setting value 318 of the particular control, and buttons 314, 316, sliders, or the like for increasing 314 or decreasing 316 the setting value of the particular control receiving user interaction 400 at the touch panel display 200. The control 312 presented at the main display 300 corresponds with the touch panel control 210 at the location of a user's finger 400 on or near the surface of the touch panel display 200. For example, if a user's finger 400 is hovering over or touching a dynamic contrast control 212 at the touch panel display 200, a corresponding dynamic contrast control 312 is presented in the dedicated area 310 of the main display 300 as shown in FIG. 2. As another example, if a user's finger 400 is hovering over or touching an acoustic output control 212 at the touch panel display 200, a corresponding acoustic output control 312 is presented in the dedicated area 310 of the main display 300 as shown in FIG. 3. As another example, if a user's finger 400 is hovering over or touching an angle control 212 at the touch panel display 200, a corresponding angle control 312 is presented in the dedicated area 310 of the main display 300 as shown in FIG. 4. In an exemplary embodiment, the visual feedback 312-318 of the touch panel control 210-218 may include a positional indicator showing a position of a user's finger 400 relative the touch panel control 210-218. The positional indicator may be an icon, shape (e.g., dot, star, square, etc.), or any suitable indicator overlaid on the visual feedback 312-318. The user is able to visualize the current value 318 of the identified control 312, a positional indicator of the user's finger, and/or locations of buttons 314, 316, sliders, and the like for increasing 314, decreasing 316 or otherwise changing the setting value at the dedicated area 310 on the main display 300 such that the user may move their finger 400 to appropriate locations of the display 200 of the touch panel 150 to make setting value adjustments or other changes without having to look at the touch panel display 200.

Referring again to FIG. 1, the display system 134 may be any device capable of communicating visual information to a user. For example, a display system 134 may include a liquid crystal display, a light emitting diode display, and/or any suitable display or displays. The display system 134 can be operable to present a main display 300 of information from the signal processor 132 and/or archive 138, such as ultrasound image data 320, visual feedback 312-318 mirroring touch panel controls 210 interacted with at a separate touch panel 150, 200, and/or any suitable information. The display system 134 may include a dedicated area 310 configured to be blank when a user is not interacting with a touch panel 150 and configured to mirror touch panel controls 210 that the user is interacting with when the user is interacting with the touch panel 150.

The archive 138 may be one or more computer-readable memories integrated with the ultrasound system 100 and/or communicatively coupled (e.g., over a network) to the ultrasound system 100, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things. In various embodiments, the archive 138 stores instructions for selectively displaying mirrored 312-318 touch panel controls 210-218 based on user interactions with a touch panel 150, 200 at a dedicated area 310 of a main display 300 of a display system 134, for example.

Components of the ultrasound system 100 may be implemented in software, hardware, firmware, and/or the like. The various components of the ultrasound system 100 may be communicatively linked. Components of the ultrasound system 100 may be implemented separately and/or integrated in various forms.

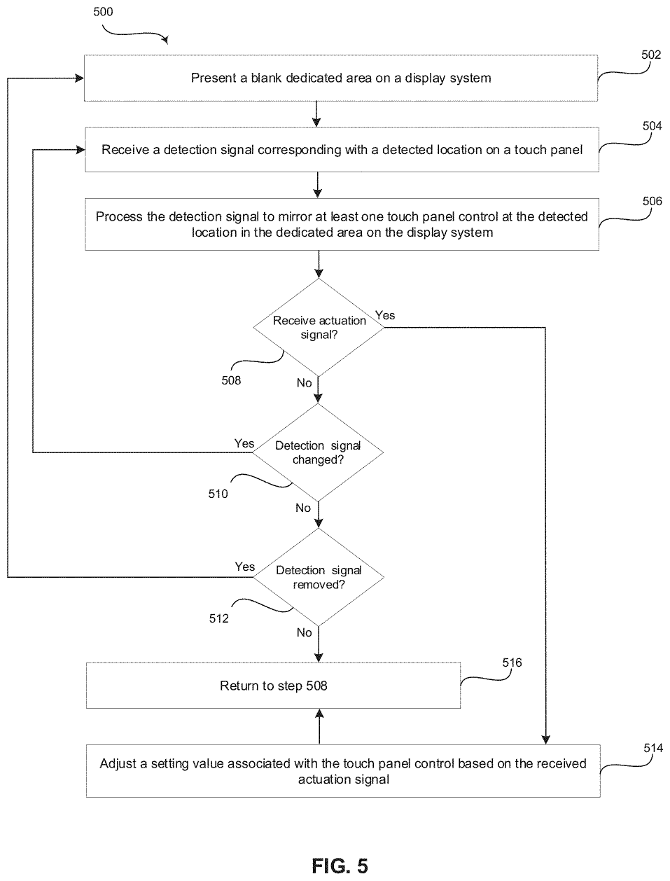

FIG. 5 is a flow chart 500 illustrating exemplary steps 502-516 that may be utilized for providing visual feedback 312-318 at a main display 300 identifying a control 312 and associated setting value 318 corresponding to user interaction 400 with a touch panel control 210-218, in accordance with various embodiments. Referring to FIG. 5, there is shown a flow chart 500 comprising exemplary steps 502 through 516. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 502, a signal processor 132 of an ultrasound system 100 may present a blank dedicated area 310 on a display system 134. For example, a main display 300 of the display system 134 of the ultrasound system 100 may include an ultrasound image display area configured to present an ultrasound image 320 and a dedicated area 310 configured to selectively present visual feedback 312-318 related to user interaction and actuation of touch panel controls 210. Prior to a user interacting with a touch panel 150 or when a user has stopped interacting with the touch panel 150, a touch panel control processor 140 of the signal processor 132 may be configured to leave the dedicated area 310 on the main display 300 blank such that the user is not distracted by the presentation of non-image display elements. As an example, prior to user interaction with the touch panel 150, such as when an ultrasound operator is manipulating the ultrasound probe 104 and reviewing the acquired ultrasound images on the display system 134, the dedicated area on the main display of the display system 134 may be left blank.

At step 504, a signal processor 132 of an ultrasound system 100 may receive a detection signal corresponding with a detected location on a touch panel 150. For example, the touch panel control processor 140 of the signal processor may receive a detection signal from a detection sensor 150a of the touch panel 150. The touch panel 150 may include a detection sensor 150a operable to detect a user touching and/or hovering over the touch panel 150. The detection sensor 150a may be a resistive sensor, capacitive sensor, infrared sensor, or any suitable sensor operable to detect a user touching and/or in close proximity to the sensor. For example, the detection 150a sensing may be performed by resistive film touch panels, surface capacitive touch panels, projected capacitive touch panels, surface acoustic wave (SAW) touch panels, optical touch panels (e.g., infrared optical imaging touch panels), electromagnetic induction touch panels, or any suitable touch panel 150. The locations on the surface of the touch panel 150 may be associated with touch panel controls 210 presented at corresponding positions on a display 200 of the touch panel 150. The detection sensor 150a may be operable to send a detection signal to a touch panel control processor 140 in response to detection of the user touching and/or hovering over the user input device 130. The detection signal may include information related to the location of the user interaction on the touch panel 150.

At step 506, the signal processor 132 of the ultrasound system 100 may process the detection signal to mirror 312-318 at least one touch panel control 210-218 at the detected location in the dedicated area 310 on the display system 134. For example, the touch panel control processor 140 of the signal processor 132 may process the detection signal received from the detection sensor 150a of the touch panel 150 at step 504 to identify the touch panel control 210-218 associated with the location of the user interaction on the touch panel 150 as identified by the detection signal. The touch panel control processor 140 may selectively present, at the dedicated area 310 in the main display 300 of the display system 134, the identified touch panel control 210-218 mirrored from the touch panel 150. For example, the touch panel 150 may present various controls 210, 212, each having an associated setting value 218 and buttons 214, 216, sliders, or the like for adjusting the setting value 218, at various locations on the touch panel 150. The touch panel control processor 140 may be configured to process the detection signal to identify the particular control 210-218 presented at the location on the touch panel 150 and present a mirrored representation 312-318 of the particular control 210-218 in the dedicated area 310 at the main display 300 of the display system 134 such that an ultrasound operator does not have to look away from the main display 300 to visualize the control 210-218 the user is interacting with at the touch panel 150. As an example, the identification 312 of the control, the setting value 318 associated with the control, and the buttons 314, 316, sliders, or the like for adjusting the setting value 318 of the control may be presented in the dedicated area 310 of the main display 300 of the display system 134 in substantially a same manner as presented at the touch panel 150 to provide visual feedback 312-318 to an ultrasound operator such that the operator is able to interact with the buttons 214,216, sliders, and the like at the touch panel 150 to adjust the setting value 218,318 of the control 210,212,312 without looking at the touch panel 150. In an exemplary embodiment, the visual feedback 312-318 of the touch panel control 210-218 may include a positional indicator showing a position of a user's finger 400 relative the touch panel control 210-218.

At step 508, the signal processor 132 of the ultrasound system 100 may determine whether an actuation signal has been received. For example, the touch panel control processor 140 of the signal processor 132 may determine whether an actuation signal was received from the actuation sensor 150b of the touch panel 150. The actuation sensor 150b may be a resistive sensor, capacitive sensor, infrared sensor, or any suitable sensor operable to detect a user depressing the sensor. For example, the actuation 150b sensing may be performed by resistive film touch panels, surface capacitive touch panels, projected capacitive touch panels, surface acoustic wave (SAW) touch panels, optical touch panels (e.g., infrared optical imaging touch panels), electromagnetic induction touch panels, or any suitable touch panel 150. The actuation sensor 150b may be operable to detect an actuation of the touch panel 150. For example, the actuation sensor 150b may provide the signal processor 132 with an actuation signal corresponding with the depression of a location on the touch panel 150 surface. The actuation signal may correspond with the actuation of a button 214, 216, slider, or the like at the depressed location to adjust a setting value 218, 318 of the control 210, 212. If the touch panel control processor 140 received an actuation signal from the actuation sensor 150b of the touch panel 150, the process proceeds to step 514. If the touch panel control processor 140 has not received an actuation signal from the actuation sensor 150b of the touch panel 150, the process proceeds to step 510.

At step 510, the signal processor 132 of the ultrasound system 100 may determine whether the detection signal has changed. For example, the touch panel control processor 140 may actively monitor the detection signal received from the detection sensor 150a of the touch panel 150 to determine whether a user is still hovering over and/or touching the touch panel 150. If the detection signal has not changed, indicating that the detection sensor 150a is still detecting a user in a defined proximity of the touch panel 150, the process may proceed to step 512. If the detection signal has changed (e.g., the detection sensor 150a is no longer detecting a user at a same location corresponding with a same touch panel control 210-218 presented at the display 200 of the touch panel 150 and is instead detecting a user at a different location corresponding with a different touch panel control 210-218 presented at the display 200 of the touch panel 150), the process may proceed to step 504 based on the different detection signal.

At step 512, the signal processor 132 of the ultrasound system 100 may determine whether the detection signal is no longer being received. For example, the touch panel control processor 140 may actively monitor the detection signal received from the detection sensor 150a of the touch panel 150 to determine whether a user is still hovering over and/or touching the touch panel 150. If the detection signal is still present, indicating the detection sensor 150a is still detecting a user in a defined proximity of the touch panel 150, the process may proceed to step 516. If the detection signal is no longer being received, indicating that the detection sensor 150a is no longer detecting a user in a defined proximity of the touch panel 150, the process may proceed to step 502 after no detection signal is received for a predetermined period of time (e.g., after 1-5 seconds without user interaction at the touch panel 150). For example, after an ultrasound operator adjusts setting values 218, 318 via touch panel controls 210-218 of the touch panel 150 and removes their finger 400 from the touch panel 150, such as to resume manipulation of the ultrasound probe 104 and/or review of the acquired ultrasound images 320, the touch panel control processor 140 may remove the displayed touch panel control and present nothing in the dedicated area 310 of the main display 300 of the display system 134 after the predetermined period of time at step 502.

At step 514, the signal processor 132 of the ultrasound system 100 may adjust a setting value 218, 318 associated with the touch panel control 210-218 based on the received actuation signal. For example, the touch panel control processor 140 of the signal processor 132 may process the actuation signal to implement the setting value adjustment corresponding with the actuated touch panel control 210-218. The touch panel control processor 140 dynamically updates the presentation of the setting value 318 at the dedicated area 310 of the main display 300 of the display system 134. For example, the setting value may change between on and off, to a different level (e.g., low, mid, high), to a different numerical value, and/or to a different dB, Hz, kHz, percentage, degree, or the like.

At step 516, the process may return to step 508 until the signal processor 132 of the ultrasound system 100 receives an additional actuation signal at step 508, a change in detection signal at step 510, or stops receiving the detection signal at step 512.

Aspects of the present disclosure provide a method 500 and system 100 for detecting user interaction 400 with a touch panel control 210-218 of an ultrasound system 100 and providing visual feedback 312-318 at a main display 134, 300 identifying the control 312 and associated setting value 318 corresponding to the user interaction 400 with the touch panel control 210-218. In accordance with various embodiments, the method 500 may comprise presenting 502, by at least one processor 132, 140 of an ultrasound system 100, an ultrasound image display area 320 and a dedicated area 310 on a main display 300 of a display system 134. The method 500 may comprise receiving 504, by the at least one processor 132, 140, a detection signal from a touch panel 150 of the ultrasound system 100. The detection signal may be provided by the touch panel 150 in response to a detection of a user 400 at a proximity to the touch panel 150. The detection signal may correspond with a location on the touch panel 150. The location on the touch panel 150 may be associated with a touch panel control 210-218 presented at the location on the touch panel 150. The method 500 may comprise processing 506, by the at least one processor 132, 140, the detection signal to display a mirrored representation 312-318 of the touch panel control 212-218 presented at the location on the touch panel 150 in the dedicated area 310 of the main display 300 of the display system 134. The method 500 may comprise receiving 508, by the at least one processor 132, 140, an actuation signal from the touch panel 150. The actuation signal may be provided by the touch panel 150 in response to a user selection at the touch panel 150. The method 500 may comprise processing 514, by the at least one processor 132, 140, the actuation signal to adjust a setting value 218, 318 of the ultrasound system 100 associated with the touch panel control 212-218.

In a representative embodiment, the mirrored representation 312-318 of the touch panel control 212-218 in the dedicated area 310 of the main display 300 of the display system 134 includes a dynamically updating positional indicator showing a current position of the user at the proximity of the touch panel 150 relative the touch panel control 212-218. In an exemplary embodiment, the proximity of the user 400 to the touch panel is one or both of the user 400 hovering over the touch panel 150, or the user 400 touching the touch panel 150. In various embodiments, the dedicated area 310 on the main display 300 of the display system 134 is blank prior to receiving 504 the detection signal and after a predetermined period of time after the detection signal is no longer received 512. In certain embodiments, the touch panel control 212-218 and the mirrored representation 312-318 of the touch panel control 212-218 comprises an identification 212, 312 of the touch panel control, the setting value 218, 318 of the ultrasound system 100 associated with the touch panel control 212-218, and a mechanism 214, 216, 314, 316 operable to adjust the setting value 218, 318. In a representative embodiment, the mechanism 214, 216, 314, 316 operable to adjust the setting value 218, 318 is one or both of at least one button 214, 216, 314, 316 and a slider. In an exemplary embodiment, the processing 514, by the at least one processor 132, 140, the actuation signal to adjust the setting value 218, 318 of the ultrasound system 100 associated with the touch panel control 212-218 comprises dynamically updating the mirrored representation 312-318 of the touch panel control 212-218 to reflect the setting value 218, 318 after adjustment.

Various embodiments provide an ultrasound system 100 for detecting user interaction 400 with a touch panel control 210-218 and providing visual feedback 312-318 at a main display 300 identifying the control 312 and associated setting value 318 corresponding to the user interaction 400 with the touch panel control 210-218. The ultrasound system 100 may comprise a display system 134, a touch panel 150, at least one processor 132, 140. The display system 134 may comprise a main display 300 having an ultrasound image display area 320 and the dedicated area 310. The touch panel 150 may be operable to provide a detection signal in response to a detection of a user 400 at a proximity to the touch panel 150. The detection signal may correspond with a location on the touch panel 150. The location on the touch panel 150 may be associated with a touch panel control 212-218 presented at the location on the touch panel 150. The touch panel 150 may be operable to provide an actuation signal in response to a user selection at the touch panel 150. The at least one processor 132, 140 may be configured to receive the detection signal from the touch panel 150. The at least one processor 132, 140 may be configured to process the detection signal to display a mirrored representation 312-318 of the touch panel control 212-218 presented at the location on the touch panel 150 in the dedicated area 310 of the main display 300 of the display system 134. The at least one processor 132, 140 may be configured to receive the actuation signal from the touch panel 150. The at least one processor 132, 140 may be configured to process the actuation signal to adjust a setting value 218, 318 of the ultrasound system 100 associated with the touch panel control 212-218.

In an exemplary embodiment, the at least one processor 132, 140 is configured to present a dynamically updating positional indicator showing a current position of the user 400 at the proximity of the touch panel 150 relative the touch panel control 212-218 with the mirrored representation 312-318 of the touch panel control 212-218 in the dedicated area 310 of the main display 300 of the display system 134. In various embodiments, the proximity of the user 400 to the touch panel 150 is one or both of the user 400 hovering over the touch panel 150, or the user 400 touching the touch panel 150. In certain embodiments, the dedicated area 310 on the main display 300 of the display system 134 is blank prior to receiving the detection signal and after a predetermined period of time after the detection signal is no longer received. In a representative embodiment, the touch panel control 212-218 and the mirrored representation 312-318 of the touch panel control 212-218 comprises an identification 212, 312 of the touch panel control, the setting value 218, 318 of the ultrasound system 100 associated with the touch panel control 212-218, and a mechanism 214, 216, 314, 316 operable to adjust the setting value 218, 318. In an exemplary embodiment, the mechanism 214, 216, 314, 316 operable to adjust the setting value 218, 318 is one or both of at least one button 214, 216, 314, 316 and a slider. In various embodiments, the at least one processor 132, 140 is configured to dynamically update the mirrored representation 312-318 of the touch panel control 212-218 to reflect the setting value 218, 318 after adjustment.

Certain embodiments provide a non-transitory computer readable medium having stored thereon, a computer program having at least one code section. The at least one code section is executable by a machine for causing an ultrasound system 100 to perform steps 500. The steps 500 may comprise presenting 502 an ultrasound image display area 320 and a dedicated area 310 on a main display 300 of a display system 134. The steps 500 may comprise receiving 504 a detection signal from a touch panel 150 of the ultrasound system 100. The detection signal may be provided by the touch panel 150 in response to a detection of a user 400 at a proximity to the touch panel 150. The detection signal may correspond with a location on the touch panel 150. The location on the touch panel 150 may be associated with a touch panel control 212-218 presented at the location on the touch panel 150. The steps 500 may comprise processing 506 the detection signal to display a mirrored representation 312-318 of the touch panel control 212-218 presented at the location on the touch panel 150 in the dedicated area 310 of the main display 300 of the display system 134. The steps 500 may comprise receiving 508 an actuation signal from the touch panel 150. The actuation signal may be provided by the touch panel 150 in response to a user selection at the touch panel 150. The steps 500 may comprise processing 514 the actuation signal to adjust a setting value 218, 318 of the ultrasound system 100 associated with the touch panel control 212-218.

In various embodiments, the mirrored representation 312-318 of the touch panel control 212-218 in the dedicated area 310 of the main display 300 of the display system 134 includes a dynamically updating positional indicator showing a current position of the user 400 at the proximity of the touch panel 150 relative the touch panel control 212-218. In certain embodiments, the proximity of the user 400 to the touch panel 150 is one or both of the user 400 hovering over the touch panel 150, or the user 400 touching the touch panel 150. In a representative embodiment, the dedicated area 310 on the main display 300 of the display system 134 is blank prior to receiving 504 the detection signal and after a predetermined period of time after the detection signal is no longer received 512. In an exemplary embodiment, the touch panel control 212-218 and the mirrored representation 312-318 of the touch panel control 212-218 comprises an identification 212, 312 of the touch panel control 212-218, the setting value 218, 318 of the ultrasound system 100 associated with the touch panel control 212-218, and one or both of at least one button 214, 216, 314, 316 and a slider operable to adjust the setting value 218, 318. In various embodiments, the processing 514 the actuation signal to adjust the setting value 218, 318 of the ultrasound system 100 associated with the touch panel control 212-218 comprises dynamically updating the mirrored representation 312-318 of the touch panel control 212-218 to reflect the setting value 218, 318 after adjustment.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” and/or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for detecting user interaction with a touch panel control of an ultrasound system and providing visual feedback at a main display identifying the control and associated setting value corresponding to the user interaction with the touch panel control.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

1. A method comprising:
presenting, by at least one processor of an ultrasound system, an ultrasound image display area and a dedicated area on a main display of a display system, wherein the dedicated area is non-overlapping with the ultrasound image display area;
receiving, by the at least one processor, a detection signal from a touch panel of the ultrasound system, wherein:
the detection signal is provided by the touch panel in response to a detection of a user at a proximity to the touch panel, and
the detection signal corresponds with a location on the touch panel, the location on the touch panel associated with a touch panel control presented at the location on the touch panel;
processing, by the at least one processor, the detection signal to display a mirrored representation of the touch panel control presented at the location on the touch panel in the dedicated area of the main display of the display system;
receiving, by the at least one processor, an actuation signal from the touch panel, wherein the actuation signal is provided by the touch panel in response to a user selection at the touch panel; and
processing, by the at least one processor, the actuation signal to adjust a setting value of the ultrasound system associated with the touch panel control
wherein the dedicated area on the main display of the display system is blank prior to receiving the detection signal and after a predetermined period of time after the detection signal is no longer received.

2. The method of claim 1, wherein the mirrored representation of the touch panel control in the dedicated area of the main display of the display system includes a dynamically updating positional indicator showing a current position of the user at the proximity of the touch panel relative the touch panel control.

3. The method of claim 1, wherein the proximity of the user to the touch panel is one or both of:
the user hovering over the touch panel, or
the user touching the touch panel.

4. The method of claim 1, wherein the dedicated area on the main display of the display system is below the ultrasound image display area.

5. The method of claim 1, wherein the touch panel control and the mirrored representation of the touch panel control comprises an identification of the touch panel control, the setting value of the ultrasound system associated with the touch panel control, and a mechanism operable to adjust the setting value.

6. The method of claim 5, wherein the mechanism operable to adjust the setting value is one or both of at least one button and a slider.

7. The method of claim 1, wherein the processing, by the at least one processor, the actuation signal to adjust the setting value of the ultrasound system associated with the touch panel control comprises dynamically updating the mirrored representation of the touch panel control to reflect the setting value after adjustment.

8. An ultrasound system comprising:
a display system comprising a main display having an ultrasound image display area and a dedicated area, wherein the dedicated area is non-overlapping with the ultrasound image display area;
a touch panel operable to:
provide a detection signal in response to a detection of a user at a proximity to the touch panel, the detection signal corresponding with a location on the touch panel, the location on the touch panel associated with a touch panel control presented at the location on the touch panel, and
provide an actuation signal in response to a user selection at the touch panel; and
at least one processor configured to:
receive the detection signal from the touch panel,
process the detection signal to display a mirrored representation of the touch panel control presented at the location on the touch panel in the dedicated area of the main display of the display system,
receive the actuation signal from the touch panel, and
process the actuation signal to adjust a setting value of the ultrasound system associated with the touch panel control,
wherein the dedicated area on the main display of the display system is blank prior to receiving the detection signal and after a predetermined period of time after the detection signal is no longer received.

9. The ultrasound system of claim 8, wherein the at least one processor is configured to present a dynamically updating positional indicator showing a current position of the user at the proximity of the touch panel relative the touch panel control with the mirrored representation of the touch panel control in the dedicated area of the main display of the display system.

10. The ultrasound system of claim 8, wherein the proximity of the user to the touch panel is one or both of:
the user hovering over the touch panel, or
the user touching the touch panel.

11. The ultrasound system of claim 8, wherein the dedicated area on the main display of the display system is below the ultrasound image display area.

12. The ultrasound system of claim 8, wherein the touch panel control and the mirrored representation of the touch panel control comprises an identification of the touch panel control, the setting value of the ultrasound system associated with the touch panel control, and a mechanism operable to adjust the setting value.

13. The ultrasound system of claim 8, wherein the mechanism operable to adjust the setting value is one or both of at least one button and a slider.

14. The ultrasound system of claim 8, wherein the at least one processor is configured to dynamically update the mirrored representation of the touch panel control to reflect the setting value after adjustment.

15. A non-transitory computer readable medium having stored thereon, a computer program having at least one code section, the at least one code section being executable by a machine for causing an ultrasound system to perform steps comprising:
presenting an ultrasound image display area and a dedicated area on a main display of a display system, wherein the dedicated area is non-overlapping with the ultrasound image display area;
receiving a detection signal from a touch panel of the ultrasound system, wherein:
the detection signal is provided by the touch panel in response to a detection of a user at a proximity to the touch panel, and
the detection signal corresponds with a location on the touch panel, the location on the touch panel associated with a touch panel control presented at the location on the touch panel;
processing the detection signal to display a mirrored representation of the touch panel control presented at the location on the touch panel in the dedicated area of the main display of the display system;
receiving an actuation signal from the touch panel, wherein the actuation signal is provided by the touch panel in response to a user selection at the touch panel; and
processing the actuation signal to adjust a setting value of the ultrasound system associated with the touch panel control,
wherein the dedicated area on the main display of the display system is blank prior to receiving the detection signal and after a predetermined period of time after the detection signal is no longer received.

16. The non-transitory computer readable medium of claim 15, wherein the mirrored representation of the touch panel control in the dedicated area of the main display of the display system includes a dynamically updating positional indicator showing a current position of the user at the proximity of the touch panel relative the touch panel control.

17. The non-transitory computer readable medium of claim 15, wherein the proximity of the user to the touch panel is one or both of:
the user hovering over the touch panel, or
the user touching the touch panel.

18. The non-transitory computer readable medium of claim 15, wherein the dedicated area on the main display of the display system is below the ultrasound image display area.

19. The non-transitory computer readable medium of claim 15, wherein the touch panel control and the mirrored representation of the touch panel control comprises an identification of the touch panel control, the setting value of the ultrasound system associated with the touch panel control, and one or both of at least one button and a slider operable to adjust the setting value.

20. The non-transitory computer readable medium of claim 15, wherein the processing the actuation signal to adjust the setting value of the ultrasound system associated with the touch panel control comprises dynamically updating the mirrored representation of the touch panel control to reflect the setting value after adjustment.
